(19) United States
(12) Patent Application Publication
(10) Pub. No.: US 2006/0034947 A1
(43) Pub. Date: Feb. 16, 2006

(54) Erythritol compositions for nasopharynx cleansing

(76) Inventor: James B. Burr, Provo, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/143,146

(22) Filed: Jun. 2, 2005

Related U.S. Application Data
(60) Provisional application No. 60/521,599, filed on Jun. 2, 2004.

(51) Int. Cl. A61K 33/14; A61K 31/045; A61K 36/752

(52) U.S. Cl. 424/680; 514/738; 424/736

(57) ABSTRACT

A cleansing composition for use in cleansing a subject's nasopharynx can include an effective amount of erythritol admixed with an acceptable carrier. The cleansing composition can be configured to be capable of being applied to the nasopharynx through the subject's nostril. Additionally, a method of cleansing a subject's nasopharynx can include nasally administering the cleansing composition to the nasopharynx. The method can also include retaining a portion of the cleansing composition in the nasopharynx for a sufficient duration to effect the cleansing.

### CROSS-REFERENCE TO RELATED APPLICATIONS

This application claims priority to Provisional Application 60/521,599 filed Jun. 2, 2004, which is incorporated herein by reference in its entirety.

### BACKGROUND OF THE INVENTION

1. The Field of the Invention

The present invention relates to nasopharynx cleansing compositions. More particularly, this invention relates to erythritol-containing compositions that can be used to clean the nasopharynx, and treat and/or prevent upper respiratory infections.

2. The Relevant Technology

The nasopharynx is a respiratory cavity located between the nasal passageways and oropharynx. Laterally and posteriorly, the nasopharynx is also related to the deep spaces of the suprahyoid neck, particularly, the parapharyngeal, masticator and carotid spaces. Some organisms that cause upper respiratory infections are carried primarily into the nasopharynx via the nasal passageways during respiration. The temperature and wet conditions of the nasopharynx as well as the abundant supply of inspired micro-particles provides an almost ideal habitat for pathogenic bacteria to grow. A person carrying these pathogenic bacteria may exhibit signs of an upper respiratory tract infection, which can include excessive mucous, coughing, headache, and other symptoms. Also, a person having pathogens in their nasopharynx may have no symptoms, but can still infect others with the pathogen and resulting illness.

Bacteria that normally grow in the nasal and other respiratory tract cavities can be identified by a nasopharyngeal culture. Common bacteria that can be found in such a culture include, for example, non-hemolytic streptococci, alpha-hemolytic streptococci, some Neisseria species, and some types of staphylococci. Sometimes the most serious of these organisms is Neisseria meningitidis, which causes meningitis or blood stream infection in infants.

Portions of the respiratory tract are lined with a number of microscopic hairs (cilia) that are constantly in motion sweeping inspired debris toward the outside of the nose. However, some particles that are breathed in, mostly bacteria or viruses, want to cling to the epithelial cells lining the passage and are not removed by the cilia. Any such attachment to the lining can induce an inflammatory response, which can include the release of histamine. Histamine can increase the permeability of small blood vessels in the area and increases the amount of plasma in the area. These physiological processes can be recognized first as a nasal discharge, then as nasal congestion, and finally as postnasal drip where the fluid drains backward into the throat. When physiological washing is ineffective, the bacteria can migrate to the sinus cavities, where they can live and propagate for extended periods of time.

The physiological reaction to bacteria accumulating in the nasopharynx can be described as a strictly defensive response in the beginning. Accordingly, the body is trying to wash the offending organisms away. If this is unsuccessful and the organism continues to reside in the nasopharynx, the outcome is usually chronic sinusitis which may result in an infected person to be predisposed to a chronic sore throat, or even asthma, because of the constant irritation of the contaminated postnasal drip.

Additionally, decongestant drugs are potentially harmful because they can block the washing effort by constricting the blood vessels, reducing the plasma leakage, and thereby reduce the amount of washing in the nasopharynx. Also, these drugs can neutralize the histamine that causes the physiological washing response. As such, chronic sinus infections can be caused by inhibiting the washing response through the use of prescription or over-the-counter drugs.

In contrast to decongestant drugs, various attempts to implement nasopharynx washing techniques have been attempted. One such attempt included saline solutions because it is known that a salt water solution can be very cleansing to the nose, nostrils, and nasal cavities. Also, the percentage of salt in such saline solutions is very important because too little salt has no effect, and too much salt can paralyze the very important action of nasal cilia.

Additionally, some of the bacteria living in the nasopharynx area have become resistant to the antibiotics which are commonly used to treat upper respiratory infections. As a consequence, antibiotics may not be helpful in removing harmful bacteria from the nasopharynx.

Recently, erythritol has been shown to be effective in inhibiting bacteria growth. Erythritol is a polyol that occurs naturally in a wide variety of fruits and vegetables, and has functional properties similar to sucrose, though its caloric content is close to zero. Also, erythritol does not cause digestive intolerance, unlike many other polyol sweeteners. Although erythritol is chemically a polyol, it is such a small molecule that it behaves very differently from other polyols in the way it passes through the digestive system. The proximal intestine absorbs these types of molecules at a rate related to their molecular size. Therefore, erythritol, a four carbon sugar, is absorbed at a much faster rate than other sugars with larger molecules such as mannitol or glucose. Some larger polyols have slow rates of absorption from the intestine, which leads to osmotic diarrhea. Studies have indicated that erythritol, even when it comprises 20% of the diet, does not cause diarrhea. Additionally, because of erythritol's low glycemic index, it does not affect blood glucose levels and so can safely be consumed by people with diabetes.

These unique properties have resulted in erythritol being used as a sugar replacement, and the FDA has awarded erythritol GRAS (generally recognized as safe) status. Erythritol is currently used as a sugar replacement in candies, syrups, low calorie beverages, and is increasingly being used in the pharmaceutical and dental industries. Erythritol does not promote tooth decay because bacteria cannot metabolize it. In fact, erythritol is capable of partially inhibiting oral bacteria in their ability to ferment certain sugars.

Thus, what is needed is an erythritol composition that is configured for being administered to the nasopharynx via a nasal passageway. Also, it would be beneficial to have methods of administering erythritol for cleansing the nasopharynx.

### BRIEF SUMMARY OF EMBODIMENTS OF THE INVENTION

Generally, an embodiment of the present invention is an erythritol composition for use in cleansing a subject's nasopharynx. The composition includes an effective amount of erythritol that is capable of cleansing the subject's nasopharynx. Additionally, the composition includes an acceptable carrier admixed with the effective amount of erythritol. The erythritol composition is configured into a solution that can be administered into the nasopharynx.

Another embodiment of the present invention is a method of cleansing a subject's nasopharynx. The method includes administering a cleansing composition into the subject's nasopharynx, which can be via the subject's nostril. The cleansing composition includes an effective amount of erythritol admixed into an acceptable carrier. Additionally, the method can include retaining a portion of the administered cleansing composition in the nasopharynx for a duration sufficient to effect cleansing of the nasopharynx.

These and other embodiments and features of the present invention will become more fully apparent from the following description and appended claims, or may be learned by the practice of the invention as set forth hereinafter.

### DETAILED DESCRIPTION OF EMBODIMENTS OF THE INVENTION

Embodiments of the present invention include nasopharynx cleansing compositions and associated methods of using the compositions. As the present invention is disclosed and described, it is to be understood that this invention is not limited to the particular materials, combinations, and methods disclosed herein. Accordingly, this disclosure is extended to equivalents of the materials, combinations, and methods as would be recognized by one of ordinary skill in the relevant arts. It should be understood that terminology employed herein is used for the purpose of describing particular embodiments only and is not intended to be limiting.

As used herein, the term “erythritol” refers to “erythritol” and amounts of “erythrose” that can convert to an equivalent amount of erythritol. Accordingly, “erythritol” can include erythritol and/or erythrose as well as the various isomeric forms and/or salts thereof.

As used herein, the term “non-cariogenic” refers to sweeteners which are not able to be metabolized by microbes and therefore do not contribute to the formation of dental caries. For example, erythritol is a non-cariogenic sweetener.

As used herein, the term “nasopharynx” is meant to include the respiratory passage between the nasal cavity and the throat, or more particularly the section of the pharynx between the nasal cavity and the oropharynx. As such, the nasopharynx is part of the respiratory tract.

As used herein, the term “acceptable carrier” is meant to refer to any safe and effective materials for use in the compositions of the present invention that can carry and deliver erythritol. Such materials include alkali metal salts, saline solutions, water, polysaccharides, acidic compounds, buffering agents, thickeners, humectants, flavorants, sweetening agents, and the like. Additionally, an acceptable carrier can be part of a composition that retains the erythritol during storage, where the carrier and erythritol can be dissolved into an aqueous solution prior to nasopharynx irrigation.

As used herein, the term “flavoring agent” or “flavorants” is meant to refer to flavor essences and equivalent synthetic materials which are added to flavor the composition. The flavoring agent can also include specific materials which are added to provide a warming or cooling sensation.

As used herein, the term “effective amount” refers to the minimal amount through the maximal amount of a substance or agent, which is sufficient to achieve a desired effect. For example, an effective amount of erythritol in a cleansing composition would be the minimum amount that provides the desired physiological effect such as cleansing the nasopharynx cavity, inhibiting bacteria growth, inhibited bacteria production, flushing the nasopharynx, and treating and/or preventing upper respiratory infections. Accordingly, an “effective amount” should include a safe and effective amount, which is meant to include an amount of an agent (e.g., erythritol) high enough to significantly improve the condition to be treated, but low enough to avoid serious side effects (at a reasonable benefit/risk ratio), within the scope of sound medical/dental judgment. The safe and effective amount of the agent may vary with the particular condition being treated, the age and physical condition of the patient being treated, the severity of the condition, the duration of treatment, the nature of concurrent therapy, the specific form of the source employed, and the particular carrier from which the agent is applied.

As used herein, the term “container” as described herein, means a jar, cup, can, tube, tub, pump, bottle, sprayer, or any other composition holding or dispensing means.

Concentrations, amounts, particles sizes or other numerical data may be presented in a range format. It is to be understood that such a range format is used merely for convenience and brevity and should be interpreted flexibly to include not only the numerical values explicitly recited as the limits of the ranges, but also to include all the individual numerical values or sub-ranges encompassed within that range as if each numerical value and sub-range is explicitly recited. For example, the concentration of erythritol can range at from about 1% to about 80% by weight of the total composition, where such range should be interpreted to include not only the explicitly recited limits, but also to include sub-ranges such as, for example, from about 20% to about 30% by weight as well as other sub-ranges. Also, the enumeration of such ranges should be interpreted to include such individual concentrations such as, for example, 10%, 25%, 40%, and 50% by weight as well as sub-ranges between these individual compositions. This interpretation should apply regardless of the breadth of the range or the characteristic being described, and should apply to ranges having both upper and lower numerical values as well as open-ended ranges reciting only one numerical value.

As used herein, percentages and ratios are by weight of total composition, unless otherwise indicated.

Additionally, the nasopharynx cleansing composition is described herein by enumeration of various examples of components as well as various examples of component concentrations. However, it should be recognized that these are merely illustrated for example purposes and should not be interpreted to limit the components to the explicitly enumerated examples, but should be extended to equivalents in the art known to have similar functionalities. Also, it should be recognized that the concentrations of these components are given only for example, and the actual concentration of any of the components can vary from the values exemplified because the specific components and associated concentrations within a composition can vary the amounts of the other components. Thus, the exemplified components and concentrations are provided only as indicators of the types and approximate amounts of components that can be included within the nasopharynx cleansing compositions of the present invention.

An embodiment of the present invention is a nasopharynx cleansing composition for use in a unique method of cleaning the nasopharynx in order to reduce the number of bacteria residing there. The reduction of nasopharyngeal bacteria can decrease the occurrence of upper respiratory infections such as otitis and sinusitis. Additionally, the severity of upper respiratory triggered asthma can be dramatically reduced by decreasing the number of bacteria in the nasopharynx.

In accordance with the foregoing, the present invention provides a cleansing composition intended for cleaning a subject's nasopharynx as well as the subject's nasal cavity and nostrils. The cleansing composition includes an effective amount of erythritol that is capable of cleansing the subject's nasopharynx. Additionally, the composition includes an acceptable carrier admixed with the effective amount of erythritol. The erythritol composition is configured into a solution that can be administered into the nasopharyx through the subject's nostril and nasal cavity.

An embodiment of the present invention is a cleansing composition for use in treating and/or preventing upper respiratory tract infections. Accordingly, the cleansing composition can treat and/or prevent otitis media and/or sinusitis. Additionally, the cleansing composition can be used for treating and/or preventing ear aches that are associated with upper respiratory tract infections. Moreover, the cleansing composition can be used to cleanse the nasopharynx of people who experience recurring upper respiratory infections. As such, the cleansing composition can reduce the recurrences of these infections.

Erythritol is a polyol sugar alcohol that is commonly used in baking and as a sweetener. Additionally, erythritol can provide a therapeutic effect, such as a cleansing or antibacterial effect. Erythritol can treat and/or prevent respiratory infections by neutralizing bacteria and/or aiding in the drainage of foreign substances from the nasal cavity and nasopharynx. For example, a composition in accordance with the present invention can include erythritol at a concentration of from about 1% to about 80%, more preferably from about 5% to about 30%, and most preferably from about 10% to about 20% by weight. Also, up to pure erythritol can be provided for dilution into a nasally administerable form. In any event, the cleansing composition includes an effective amount of erythritol.

Additionally, a wide variety of salts can be included in the present invention, especially when the acceptable carrier has a saline component. Some examples of salts that are suitable for use in the nasopharynx cleansing composition include alkali metal salts of acetates, ammonium sulfates, bromides, chlorides, chromates, citrates, dithionates, fluorosilicates, tartrates, fluorides, formates, iodides, nitrates, phenol sulfates, salicylates, gluconates, succinates, glycerophosphates, lactates, and the like. The salt can be present at a concentration range of from about 0.35% to about 3% by weight, more preferably about 0.40% to about 10%, and most preferably about 0.45% to about 0.85% by weight. [PLEASE CONFIRM RANGES.]

The nasopharynx cleansing composition can also include water. When water is used in the present invention, the water preferably is of low particulate content and free of organic impurities. The amount of water in a composition should be considered to be not only that added as free water, but also water which is introduced with other materials, such as with sorbitol, silica, surfactant, or other components such as salts or in saline solutions. Water can be included up to about 99% of the composition.

Also, the water can be present as a saline solution formed with a salt such as an alkali metal salt. The saline solution can include an alkali metal salt such as sodium chloride in the range of from about 0.45% to about 3% by weight of the composition. However, lower or higher concentrations of salt in a saline solution can be used depending on the whether the cleansing composition is configured to be hypotonic, isotonic, or hypertonic.

The nasopharynx cleansing composition may be a single-phase composition or may be a combination of two or more compositions delivered in various phases. The composition is a product that, in the ordinary course of usage, is not intentionally swallowed for purposes of systemic administration of particular therapeutic agents, but rather, is passed through or retained in the nasopharynx cavity and other portions of the respiratory tract. Also, it may be beneficial for the deliverable form of a nasopharynx cleansing composition to be a solution that is capable of being injected through a nostril into the nasopharynx, and have a consistency that can aid in removing foreign substances therefrom.

In order to enhance the properties of the nasopharynx cleansing composition, a thickening material or binder can be included to ensure that the storable or final composition has the desired properties. Such thickeners include carboxyvinyl polymers, polyhydroxyacids, alginates, polyacrylic acids, pentosans, polysulfates, polyorthoesters, and polysaccharides. For example, the thickeners can include celluloses, amyloses, inulins, chitins, chitosans, amylopectins, glyco-ens, pectins, hemicelluloses, glucomaimans, galactoglucomannans, xyloglucans, methylglucuronoxylans, arabinoxylans, methyl glucuronoarabinoxylans, glycosaminoglycans, chondroitins, hyaluronic acids, and alginic acids. Some more specific examples of thickening agents include, carrageens, carboxymethylcellulose, hydroxyethyl cellulose, and water soluble salts of cellulose ethers such as sodium carboxymethylcellulose and sodium hydroxyethyl cellulose.

Also, natural gums such as gum karaya, xanthum gum, gum arabic, and gum tragacanth can be used as part of the thickening agent to further modify the characteristics of the nasopharynx composition. Specific examples of thickening gum components can include sodium alginate, ammonium alginate, sodium calcium alginate, calcium alginate, potassium alginate, esters of alginic acid, propyl glycerol alginate, and the like. For example, the thickening agent can be included at a concentration of up to 50% by weight, more preferably about 0.5% to about 25%, and most preferably about 1% to about 10% by weight. [PLEASE CONFIRM.]

Plasticizers can also be combined into a nasopharynx cleansing composition to aid in providing a stable composition for storage or administration, and especially when a thickener is included. Some examples of plasticizers include glyceryl triacetate, acetylated monoglyceride, glyceryl tributyrate, ethyl laurate, ethyl acetoacetate, diethyl tartrate, ethyl or butyl lactates, diethyl malate, ethyl oleate, castor oil, succinylated monoglycerides, and the like. For example, the plasticizer can be included from up to about 5%, more preferably about 0.5% to about 3%, and most preferably about 0.75% to about 2% by weight. [PLEASE CONFIRM.]

Various oils can also be included in the nasopharynx cleansing composition. Some examples of oils can include the hydrogenated vegetable oils such as hydrogenated palm oil, hydrogenated soybean oil, hydrogenated cotton seed oil, and various other hydrogenated vegetable oils.

Additionally, in order to maintain a favorable consistency of the nasopharynx cleansing composition, a humectant can be included. This is because humectants can prevent the composition from drying out or hardening upon exposure to air, which can commonly occur during storage. Some examples of humectants can include glycerin, sorbitol, polyethylene glycol, propylene glycol, and other edible polyhydric alcohols. For example, the humectant can be included up to about 10%, more preferably about 0.5% to about 5%, and most preferably about 0.75% to about 1.5% by weight. [PLEASE CONFIRM.]

The present invention may also include a bicarbonate salts to buffer the pH of the nasopharynx cleansing composition. Bicarbonate salts are soluble in water, and can release carbon dioxide into aqueous systems. Sodium bicarbonate is the preferred bicarbonate. Additionally, the nasopharynx cleansing composition may include other buffering agents. Buffering agents can be used to adjust the pH of the compositions of a range of from about pH 3 to about pH 10. Some examples of buffering agents include hydroxides, carbonates, borates, silicates, phosphates, imidazoles, and mixtures thereof. Specific examples of buffering agents include monosodium phosphate, trisodium phosphate, sodium hydroxide, potassium hydroxide, carbonate salts, sodium carbonate, imidazole, pyrophosphate salts, citric acid, and sodium citrate. For example, the buffering agent can be included up to about 10%, more preferably about 0.5% to about 5%, and most preferably about 0.75% to about 2.5% by weight. [PLEASE CONFIRM.]

The nasopharynx cleansing composition may also incorporate an acidic compound, which may be organic or inorganic. The acidic compound may be any material that will act as a proton donor capable of neutralizing a base such as a bicarbonate. Examples of some acidic compounds can include carboxylic acids, phosphoric acids, alpha-hydroxy acids, sulfonic acids, and the like. Specific examples of acids include citric acid, malic acid, alginic acid, succinic acid, lactic acid, tartaric acid, glycolic acid, adipic acid, potassium bitartrate acid, acid sodium citrate, phosphoric acid, boric acid, and acid phosphate pyrophosphate salts. Acid anhydrides and acid salts of the above acids may also be used. The acidic compound is usually present at a concentration similar [to that of] the buffering agent.

Malodor control agents can also be included in the nasopharynx cleansing composition of the present invention. This is because the foreign substances in the nasopharynx that can cause respiratory infections can also result in noxious odors during respiration. Malodor control agents include a wide variety of materials such as, for example, 5-chloro-2-(2,4-dichlorophenoxy)-phenol (“triclosan”), phthalic acid and its salts, magnesium mono-potassium phthalate, chlorhexidine, alexidine, hexetidine, sanguinarine, benzalkonium chloride, salicylanilide, domiphen bromide, cetylpyridinium chloride (“CPC”), tetradecylpyridinium chloride (“TPC”), N-tetradecyl-4-ethylpyridinium chloride (“TDEPC”), octenifine, delmopinol, octapinol, and piperidine derivatives, zinc/stannous ion agents, antibiotics, augmentin, amoxicilline, tetracycline, doxycycline, minocycline, and metronidazole, methyl salicylate, and the like. For example, the malodor agent is present at up to about 10%, more preferably about 0.5% to about 5%, and most preferably about 1% to about 2.5% by weight. [PLEASE CONFIRM.]

Another class of malodor control agents includes absorbent materials, which can absorb odor-causing substances therein. As such, the absorbent material is used to absorb, bind or otherwise complex with volatile malodor substances. Examples of such absorbent materials include talc, zeolite; cyclodextrin, silica shell, and the like.

The present nasopharynx cleansing composition can also include surfactants. Suitable surfactants are those which are reasonably stable throughout a wide pH range. The surfactant may be anionic, amphoteric, amphoteric, zwitterionic, cationic, or mixtures thereof. For example, anionic surfactants can include the water soluble salts of alkyl sulfates having from about 8 to about 20 carbon atoms in the alkyl chain (e.g., sodium alkyl sulfate), and the water soluble salts of monoglycerides sulfonates of fatty acids having from about 8 to about 20 carbon atoms such as sodium lauryl sulfate. Nonionic surfactants can be broadly designed as uncharged surfactants having a hydrophilic portion and a hydrophobic portion. The amphoteric surfactants useful in the present invention can include derivatives of aliphatic secondary and tertiary amines in which the aliphatic hydrocarbon chain can be straight chain or branched, and wherein one of the aliphatic chains contains from about 8 to about 18 carbon atoms. Also, the amphoteric surfactant can include an anionic water solubilizing group such as, for example, carboxylates, sulfonates, sulfates, phosphates, and phosphonates. For example, the surfactant can be present at up to about 5%, more preferably about 0.25% to about 2.5% and most preferably about 0.5% to about 1% by weight. [PLEASE CONFIRM.]

The nasopharynx cleansing composition can also include an antioxidant. Antioxidants are generally recognized to include vitamin E, ascorbic acid, uric acid, carotenoids, vitamin A, flavenoids, polyphenols, herbal antioxidants, melatonin, aminoindoles, lipoic acids, and the like.

Besides erythritol, the nasopharynx cleansing composition can include other non-cariogenic sweeteners. This is because some of the cleansing composition may drain into the oral cavity, where a sweetener can counteract any negative tastes associated with the drainage. Some examples of non-cariogenic sweetening agents that can be used with erythritol include xylitol, sorbitol, mannitol, maltitol, isomalt, hydrogenated starch hydrolisate, and other non-cariogenic edible polyols such as glycerol. Combinations of erythritol and xylitol can provide exceptionally effective cleansing combinations by taking advantage of the proven antibacteriological effects of xylitol as well as the benefits of erythritol described herein.

High intensity sweeteners can also be used because they are typically non-cariogenic. Some examples of high intensity sweeteners include: dipeptide based sweeteners such as L-aspartyl-L-phenylalanine methyl ester (Aspartame), L-alpha-aspartyl-N-(2,2,4,4-tetramethyl-3-thietanyl)-D-alaninamide hydrate (Alitame), and the like; saccharin and its soluble salts such as sodium or calcium saccharin salts; cyclamate salts; and other high intensity sweeteners.

The cleansing composition can include some limited amount of a cariogenic sweetener. On the other hand, some embodiments of the present invention do not include any cariogenic sweetener. This is because the inclusion of some cariogenic sweeter may drain into the mouth during use of the cleansing composition, which can result in unfavorable consequences. In any event, some examples can include monosaccharides, disaccharides, polysaccharides of ribose, glucose, mannose, galactose, fructose, dextrose, sucrose, and sugar maltose. If used, it is preferred that compositions of the present invention comprise less than about 1%, preferably less than about 0.5%, and most preferably about no cariogenic sweetener.

Flavoring agents other than sweeteners can also be included in the nasopharynx cleansing composition. Similar to the reasons that sweeteners can be included, flavoring agents can counteract negative tastes associated with any drainage into the oral cavity. Flavoring agents are well known in the art, and include synthetic flavors and/or oils and/or essences derived from plants, roots, beans, nuts, leaves, flowers, fruits and the like. Some examples of suitable flavors include lemon, orange, banana, grape, lime, apricot, grapefruit, apple, strawberry, cherry, chocolate, pineapple, coffee, cocoa, cola, peanut, almond, liquorices, cinnamon and the like. The amount of flavoring agent employed is a matter of preference, but can be used, for example, up to about 5% by weight of the composition.

Also, the flavorant can be considered to be a cooling agent. Some examples of substances that can be considered physiological cooling agents include menthol, peppermint oil, acyclic tertiary and secondary carboxamides, 3-1-methoxy propan-1,2-diol, and the like.

The cleansing composition can also include preservatives, which can act to control microbial growth in the composition as well as the site being cleansed. Some examples of the preservatives include merthiolate, benzalkonium chloride, phenylcarbinol, and the like. Attentively, a preservative can be a natural product with preservative or antimicrobial properties such as grapefruit seed extract, among others. The amount of preservative present in the composition can vary depending on type and effectiveness wherein an effective amount is preferred.

Additionally, an embodiment of the present invention is a nasopharynx composition that can be diluted with water prior to being nasally delivered. Accordingly, in order for the composition to have favorable storage characteristics, the composition can be formulated to be concentrated so that it can be diluted into a saline or aqueous solution prior to administration. Also, the composition can be formulated into a solid or powder so that it can be dissolved in water or a saline solution prior to administration. Thus, the composition can be processed into a liquid, suspension, paste, gel, powder, or solid, where such a composition can be administered as provided or diluted as desired or needed to facilitate administration. For example, a concentrated erythritol gel can be administered into a nostril so that it can be inspired into the nasopharynx.

Another embodiment of the present invention is a method of nasopharynx cleansing. Such a method can be performed in anyone whether or not they are capable of self-performing normal clearing of the nasopharynx. However, the cleansing methods of the present invention may be especially suited for subjects incapable of self-performing normal clearing of the nasopharynx. Examples of subjects incapable of self-performing nasopharynx or respiratory tract hygiene techniques include infants, toddlers, quadriplegics, and other handicapped subjects as well as any person that cannot sufficiently blow their nose or otherwise facilitate nasal cavity drainage and cleaning.

The method of nasopharynx cleansing can include administering an erythritol-containing composition in an effective amount to the nasopharynx. Such an effective amount includes a quantity sufficient to induce any of the physiological and washing processes described herein.

Another embodiment of a method includes administering an erythritol-containing composition through at least one nostril into the nasopharynx. This can also include administering the erythritol-containing composition to or through the nasal cavity. Accordingly, the cleansing composition includes an effective amount of erythritol admixed into an acceptable carrier. Additionally, the acceptable carrier, along with the cleansing composition can be configured into a solution that can be injected into the nasopharynx through a nostril. On the other hand, the erythritol can be provided as a gel, paste, powder, or solid that is configured to be dissolved into an aqueous or saline solution prior to administration. As such, the erythritol composition can be diluted prior to delivery.

The method can also include retaining some portion of the cleansing composition within the nasopharynx for a sufficient duration. Retaining some portion of the cleansing composition provides for enhancing the beneficial effects described herein. This is because the longer some portion of the composition can be retained in the nasopharynx, the longer the composition can effect the cleansing by dislodging, solubilizing, and/or draining foreign substances. However, the erythritol can be effective by essentially passing through the nasopharynx so that the erythritol is retained therein for only a short period of time. Also, such retention can enhance the function of inhibiting bacteria growth or reproduction so as to provide an antibacterial effect as described herein.

A method of cleansing the nasopharynx can also include decreasing the amount of bacteria in the nasopharynx. When the cleansing composition is within the nasopharynx, the components in the composition can have an antibacterial effect. Accordingly, the composition can be effective at inhibiting the growth or reproduction of bacteria and/or flushing bacteria from the nasopharynx. Without being bound to theory, it is though that the erythritol-containing composition can inhibit the growth of bacteria by interfering with the bacteriological life cycle, which includes inhibiting bacteria cell growth and inhibiting bacteria reproduction. In any event, the cleansing composition can reduce the bacteria count in the nasopharynx by washing or aiding any natural nasopharynx washing.

Additionally the method of cleansing the nasopharynx can include draining a portion of the cleansing composition from the nasopharynx, wherein the draining can aid in removing foreign substances from the nasopharynx. Accordingly, when the composition is administered into the nasopharynx, the composition can absorb foreign substances. This can also include loosening substances from the interior surface of the nasopharynx cavity so that the substances can be flushed out of the cavity along with the composition. The substances can include particulates of dust, pollen and the like as well as microorganisms such as bacteria.

Additionally, the route of administration can provide additional benefits. When the administration is via a nostril, that nostril can also be cleansed. This can be evident when some portion of the administered cleansing composition drains from the nostril where the composition was administered. Also, the nasal cavity can be cleansed because some portion of the composition may removed particles and/or pathogens therefrom.

As a result of cleansing the nasopharynx as described herein, the method can include treating and/or preventing upper respiratory infections. The nasopharynx can be a site that becomes frequently infected because it is exposed to microorganisms in the air. Accordingly, by washing the nasopharynx, the substances at least partially responsible for these infections can be removed. Also, by cleansing the nasopharynx on a regular basis, accumulation and retention of these substances can be diminished. Accordingly, supplying an effective amount of erythritol to the nasopharynx can aid in breaking up agglomerated substances as well as removing the substances.

Additionally, the method can include treating and/or preventing ear aches. Often, ear aches can be linked to upper respiratory tract infections. This is partly because of the proximity of the location of the ear ache relative to the nasal cavity and nasopharynx. Inflammation in the nasopharynx can extend to the medial end of the eustachian tube, creating stasis and inflammation, which, in turn, can alter the pressure within the middle ear. These changes may be either negative (most common) or positive, relative to ambient pressure. This also permits pathogenic bacteria to colonize the normally sterile middle ear space by direct extension from the nasopharynx by reflux, aspiration, or active insufflation. Accordingly, removal of such pathogens can aid in preventing and/or treating ear aches related to respiratory infections.

More particularly, by cleansing the nasopharyx, future infections can be reduced. Sometimes people who experience a respiratory tract infection can be susceptible to being plagued by multiple infections. Accordingly, the method can include inhibiting the recurrence of upper respiratory infections in subjects in need thereof. As such, the subjects in need thereof have had at least 2 upper respiratory infections in the prior 6 months.

Additionally, the administering of erythritol-containing compositions can be performed by any technique that places some portion of the composition within the nasopharynx. Some of the methods of administration can include directly applying the erythritol-containing composition into a nostril from a container. Accordingly, such a container can be configured to include an orifice that facilitates such a direct application by spray or drop. For example, a container can be fitted with a spray mechanism so that the cleansing composition can be sprayed through a nostril into the nasal cavity. Spray mechanisms can include pump sprayers as well as squeeze sprayers. The various containers that can effect such administration are well-known in the art.

The container can also be used to apply the erythritol-containing composition to an applicator. When used, the applicator then can be used to administer the erythritol-containing composition to a nostril so that it can be inhaled or drained into the nasopharynx. Some examples of applicators can include cotton tip applicator, swab, gauze, pad, and the like that can deliver liquids, suspensions, gels, pastes, and the like.

For daily cleansing, or during any application, the administering can include applying a sufficient amount of the erythritol-containing composition to provide a beneficial purpose as described herein. In some embodiments, the amount can be at least one spray of the composition in one or both nostrils. For example, the amount can be between about 2 to about 5 sprays per application. The amount can be increased or decreased as needed.

Also, the administering can be part of a therapeutic regimen performed to provide any of the beneficial purposes described herein. Such a regimen can vary depending on the desired results as well as the compositional elements in the formulation being administered. The regimen can include from one administration per day to multiple administrations per day such as, for example, between about 1 to about 7 applications per day. Alternatively, the administering regimen can be correlated to various activities that commonly occur on a daily basis. Some examples of correlations with various activities includes after eating or drinking, at the time of changing a diaper, before sleeping, after awaking, and the like.

For example, nasopharynx cleansing can be performed by administering a cleansing composition into the subject's nostril. Accordingly, the cleansing composition includes erythritol at from about 1% to about 80% by weight admixed with a saline solution. The saline solution includes sodium chloride at from about 0.45% to about 0.85% by weight, and an effective amount of one of benzalkonium chloride, phenylcarbinol or grapefruit seed extract. Also, the method includes passing the administered cleansing composition through nasopharynx to effect cleansing of the nasopharynx. Thus, the method provides cleansing to the nasopharynx so as to remove foreign substances therefrom.

The present invention is further exemplified in the following examples, which are offered by way of illustration and are not intended to limit the invention in any manner.

### EXAMPLES

### Example 1

### Erythritol Spray Solution

An erythritol-containing nasopharynx composition is formulated in accordance with the present invention. The composition is prepared by mixing 15% erythritol and 85% saline solution, where all percentages represent the amount per weight of the final solution. The saline solution is formulated to include 0.5% sodium chloride. The solution is mixed so that the components become homogeneously distributed with one another, which can be denoted as Formulation 1. The composition is then loaded into a container fitted with a spray-pump so that it can be administered as a spray.

### Example 2

### Erythritol Compositions

Erythritol compositions containing various components and associated concentrations are prepared in accordance with the procedure described in Example 1. The resulting compositions are illustrated in Table 1.

TABLE 1

| Component | % (by weight) |
|---|---|
| FORMULATION 2 | |
| Erythritol | 11 |
| Citricidal ™ (Bio/Chem Research, Petaluma, CA) | 0.5 |
| Water | 87.5 |
| 1% saline solution | 1 |
| FORMULATION 3 | |
| Erythritol | 11 |
| Citricidal ™ (Bio/Chem Research, Petaluma, CA) | 0.5 |
| Purified water | 88.5 |
| FORMULATION 4 | |
| Erythritol | 21 |
| Citricidal ™ (Bio/Chem Research, Petaluma, CA) | 0.5 |
| Phosphoric acid | 1 |
| Sodium bicarbonate | 1.5 |
| Purified water | 77 |
| FORMULATION 5 | |
| Erythritol | 20 |
| Citricidal ™ (Bio/Chem Research, Petaluma, CA) | 0.5 |
| 1% saline solution | 79.5 |
| FORMULATION 6 | |
| Erythritol | 10 |
| Carboxymethylcellulose (15,000 Mw) | 0.5 |
| Castor oil | 0.5 |
| 1% saline solution | 89 |
| FORMULATION 7 | |
| Erythritol | 10 |
| Xylitol | 5 |
| Carboxymethylcellulose (15,000 Mw) | 0.5 |
| Citricidal ™ (Bio/Chem Research, Petaluma, CA) | 0.5 |
| 1% saline solution | 84 |
| FORMULATION 8 | |
| Erythritol | 11 |
| Xylitol | 10 |
| Citricidal ™ (Bio/Chem Research, Petaluma, CA) | 0.5 |
| 1% saline solution | 78.5 |

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An erythritol composition for use in cleansing a subject's nasopharynx, the composition comprising:
an effective amount of erythritol that is capable of cleansing the subject's nasopharynx;
an acceptable carrier admixed with the effective amount of erythritol, wherein the acceptable carrier is configured to be administered into the nasopharynx.

2. An erythritol composition as in claim 1, wherein the erythritol is present at a concentration from about 1% to about 80% by weight of the composition.

3. An erythritol composition as in claim 2, wherein the carrier includes a saline solution.

4. An erythritol composition as in claim 3, wherein the saline solution includes sodium chloride at from about 0.45% to about 3%.

5. An erythritol composition as in claim 3, wherein the carrier includes an effective amount of at least one of grapefruit seed extract, benzalkonium chloride or phenylcarbonol.

6. An erythritol composition as in claim 5, wherein the carrier includes at least one component selected from the group comprised of antimicrobials, preservatives, humectants, acidic agents, buffers, flavorants, malodor agents, or non-cariogenic sweeteners, wherein the composition is substantially devoid of a cariogenic sweetener.

7. A method of cleansing a subject's nasopharynx, the method comprising:
administering a cleansing composition into the subject's nasopharynx, the cleansing composition including an effective amount of erythritol admixed into an acceptable carrier.

8. A method as in claim 7, wherein the method includes cleansing the nasopharynx.

9. A method as in claim 8, wherein the method includes decreasing the amount of bacteria in the nasopharynx.

10. A method as in claim 6, wherein the method includes draining a portion of the cleansing composition from the nasopharynx, wherein the draining effects removal of foreign substances from the nasopharynx.

11. A method as in claim 10, wherein the method includes cleansing at least one of the nostril or nasal cavity.

12. A method as in claim 10, wherein the method includes at least one of treating or preventing upper respiratory infections.

13. A method as in claim 12, wherein the method includes at least one of treating or preventing ear aches.

14. A method as in claim 12, wherein the method includes inhibiting the recurrence of upper respiratory infections in a subject in need thereof.

15. A method as in claim 14, wherein the subject in need thereof has had at least two upper respiratory infections in the prior 6 month period.

16. A method as in claim 12, further comprising diluting an erythritol composition to form a nasally administrable composition.

17. A method as in claim 7, wherein the administering is part of a dosing regimen that includes between about 1 and about 7 separate nasal injections per day.

18. A method as in claim 17, wherein at least one of the nasal injections is administered at one of after waking, after eating, at a diaper change, or before sleeping.

19. A method as in claim 18, wherein the administration is performed by one of a spray, a drop, or a swab.

20. A method of cleansing a subject's nasopharynx, the method comprising:
administering a cleansing composition into the subject's nostril, the cleansing composition including erythritol at from about 1% to about 65% by weight admixed with a saline solution, the saline solution including sodium chloride at from about 0.45% to about 3% by weight, and grapefruit seed extract at from about 0.4% to about 0.6% by weight;
passing the administered cleansing composition through the nasopharynx to effect cleansing of the nasopharynx; and
cleansing the nasopharynx so as to remove foreign substances therefrom.
